(19) United States
(12) Patent Application Publication
(10) Pub. No.: US 2022/0331490 A1
(43) Pub. Date: Oct. 20, 2022

(54) FIBROUS POLYMERIC SCAFFOLDS FOR SOFT TISSUE ENGINEERING

(71) Applicant: The Trustees of Columbia University in the City of New York, New York, NY (US)

(72) Inventors: Helen H. Lu, New York, NY (US); Christopher Z. Mosher, New York, NY (US); Romare Antrobus, New York, NY (US); James C. Hone, New York, NY (US); Theanne Schiros, New York, NY (US); Yilin Yang, New York, NY (US)

(73) Assignee: The Trustees of Columbia University in the City of New York, New York, NY (US)

(21) Appl. No.: 17/668,332

(22) Filed: Feb. 9, 2022

Related U.S. Application Data
(63) Continuation of application No. PCT/US2020/045531, filed on Aug. 7, 2020.
(60) Provisional application No. 62/885,044, filed on Aug. 9, 2019.

(51) Int. Cl. A61L 27/44

(52) U.S. Cl. CPC A61L 27/443; A61L 2430/06

(57) ABSTRACT

A fibrous polymeric scaffold for soft tissue engineering comprises electrospun fibers including a polymeric blend and graphite particles embedded therein.

This application claims priority of U.S. Provisional Application No. 62/885,044, filed Aug. 9, 2019, the contents of which are hereby incorporated by reference.

Throughout this application, various publications are referenced by author and publication date within parentheses. Full citations for these publications may be found at the end of the specification or at the end of each experimental section. The disclosures of these publications are hereby incorporated by reference into this application to describe more fully the art to which this invention pertains.

This invention was made with government support under 1644869 awarded by the National Science Foundation and W81XWH-15-1-0685 awarded by the Department of Defense Translational Research Award (ARMY/MRMC). The government has certain rights in the invention.

### TECHNICAL FIELD

This disclosure relates to a fibrous polymeric scaffold for soft tissue engineering.

### BACKGROUND

Integration of existing cartilage grafts (autologous cartilage, tissue engineered cartilage, stem cell derived cartilage, and hydrogels) with host cartilage persists as a clinical challenge. This is mainly due to the zone of chondrocyte death (ZoCD), a dense region of necrosed cells that lines the periphery of the wound edge in cartilage autografts and damaged host tissue. To overcome the ZoCD, digestive rinses (collagenase, chondroitinase ABC, and others) have been used to break down the region, and growth factors (insulin-like growth factor 1, platelet-derived growth factor, basic fibroblastic growth factor, and others) have been used to enhance chondrocyte and stem cell migration to repopulate the ZoCD. However, no robust method has been developed to overcome the ZoCD and integrate cartilage grafts with host cartilage.

Electrospinning is an established fiber fabrication method in which a polymer is dissolved in a solvent, and the resulting polymer solution is loaded into a syringe. Electrostatic forces are applied to the tip of the syringe needle, and charged polymeric fibers are ejected from the syringe at a constant rate. Electrospun fibers can be collected and used in a wide variety of applications, including but not limited to tissue engineering applications such as bone, cartilage, tendon, and ligament repair or regeneration. In such applications, a variety of growth factors, ceramic components, and other materials may be incorporated into the electrospinning solution so that resulting fibers contain advantageous material and biochemical properties.

Matrices may be fabricated via electrospinning, in which a polymer melt is ejected from an electrically charged syringe, resulting in porous, fibrous structures that can be functionalized for optimal tissue engineering outcomes.

However, no technology exists to our knowledge applying conductive polymeric fibers to soft tissue integrative repair.

### SUMMARY

In this disclosure, there is provided a product of the efforts to attain efficacy of electrospun polymer-based fibers with graphite incorporated for a scaffold to enhance chondrocyte proliferation and matrix deposition compared to graphite-free fibers, to facilitate cartilage graft integration with host cartilage and ability to support full thickness chondrocyte (FTC) viability and proliferation can be produced with polymer-based fibers with graphite.

This disclosure describes various embodiments of inventive fibrous polymeric scaffolds, such as to encase a cartilage graft and support its integration with host cartilage. Various embodiments and examples are described. For example, a fibrous polymeric scaffold preferably comprises fibers including a polymeric blend of poly(lactic-co-glycolic) acid (PLGA) and poly(ε-caprolactone) (PCL), as well as graphite nanoparticles.

Such inventive polymeric matrices can be employed in meshes, graft collars, implantable devices, scaffolds, other tissue engineering tools, etc.

Various other inventive aspects can be integrated or employed, as discussed infra.

### BRIEF DESCRIPTION OF THE FIGURES

FIGS. 1A-1D show polymeric fibers composed of a 5:1 blend of PLGA and PCL fibers containing 0, 5, 10, and 15% w/w graphite that were electrospun via apparatus shown in FIG. 2.

FIG. 2 shows a graphical representation of an apparatus employing electrospinning for generating biocompatible matrices for tissue engineering.

FIG. 3 shows a diagram of Raman spectroscopy of the polymeric fibers of FIG. 1.

FIG. 4 shows a diagram of cell numbers of the polymeric fibers of FIG. 1.

FIG. 5 shows a diagram of collagen over time of the polymeric fibers of FIG. 1.

FIG. 6 shows a diagram of Glycosaminoglycans over time of the polymeric fibers of FIG. 1.

FIG. 7 shows a flow chart for a method for generating fibrous polymeric scaffolds for tissue engineering, according to an embodiment.

FIGS. 8A-8C show the characterization of graphite-containing microfibers.

FIGS. 9A-9C show cell response on graphite microfibers.

FIGS. 10A-10C show graphite hydrogel characterization and cell response.

FIGS. 11A and 11B show enhanced cell migration and functional mechanical repair with graphite microfibers.

FIGS. 12A and 12B show growth factor delivery and cell migration.

FIGS. 13A-13C show cell response to combined therapy after direct seeding onto scaffolds.

### DETAILED DESCRIPTION

### Terms

Unless otherwise defined, all technical and scientific terms used herein have the same meaning as commonly understood by a person of ordinary skill in the art to which this disclosure belongs.

As used herein, and unless stated otherwise or required otherwise by context, each of the following terms, as used in this application, shall have the definition set forth below.

The term “a” or “an” as used herein includes the singular and the plural, unless specifically stated otherwise. Therefore, the terms “a,” “an” or “at least one” can be used interchangeably in this application.

As used herein, the term “about” or “approximately” includes ±10% from the indicated values in the range. In addition, where a parameter range is provided, all integers within that range, and tenths thereof, are also provided by this disclosure. For example, “1% to 20%” includes 1.0%, 1.1%, 1.2%, 1.3%, 1.4% etc. up to 20%.

As used herein, the term “articular cartilage” is the smooth, white tissue that covers the ends of bones where they come together to form joints and bears and distribute loads across the diarthrodial joints.

As used herein, “active agent” shall mean a component incorporated into the fibrous polymeric scaffolds, which when released over time, supports alignment, proliferation and matrix deposition of a selected cell. Examples include but are in no way limited to growth factors such as transforming growth factor-beta 3 (TGF-β3), growth/differentiation factor-5 (gdf-5), bone morphogenetic protein (BMP) 1 through 14, fibroblast growth factor (FGF) and basic fibroblast growth factor (bGF). A single active agent or a combination of active agents may be incorporated into the fibrous polymeric scaffolds of this application. By “active agent” it is also meant to include an active pharmaceutical ingredient, such as, but not limited to, an anti-inflammatory, an antibiotic or a pain medicament, added to the fibrous polymeric scaffolds to enhance treatment and/or healing of the subject upon implantation.

As used herein, “aligned fibers” shall mean groups of fibers which are oriented along the same directional axis. Examples of aligned fibers include, but are not limited to, groups of parallel fibers, etc.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment or replace any tissue, organ or function of the body. The biocompatible material can perform with an appropriate host response in a specific application and does not have toxic or injurious effects on biological systems. Nonlimiting examples of biocompatible materials include a biocompatible ceramic, a biocompatible polymer, a biocompatible hydrogel, etc.

As used herein, “biocompatible matrices” shall mean three-dimensional structures fabricated from biocompatible material. The biocompatible material can be biologically-derived or synthetic.

As used herein, “biodegradable” means that the material, once implanted into a host, will begin to degrade.

As used herein, “biomimetic” or “biomimicry” shall mean a resemblance of a synthesized material to a substance that occurs naturally in a human body and which is not rejected by (e.g., does not cause an adverse reaction in) the human body.

As used herein, “biomimic” shall mean to mimic a synthesized material to the native properties of the substance that occurs in a human body.

As used herein, “biopolymer mesh” shall mean any material derived from a biological source. Examples of a biopolymer mesh include, but are not limited to, collagen, chitosan, silk, alginate, etc.

As used herein, “calcium phosphate” shall mean a family of materials and minerals containing calcium ions (Ca²⁺) together with inorganic phosphate anions. Various crystalline phases of calcium phosphate (“Ca—P”), such as hydroxyapatite Ca₅(P0₄)30H, tricalcium phosphate (Ca₃(P0₄)₂, “TCP”), amorphous calcium phosphate (Ca₃(P0₄)₂, “ACP”), octacalcium phosphate (Ca₈H₂(P0₄)65H₂0, “OCP”), tetracalcium phosphate and carbonated or fluoridated apatite can be used. Calcium phosphate cement can be used in filling bone defects in dental and orthopedic surgery. Calcium phosphate coatings on metal implants can encourage direct bone deposition on the implants, thereby forming a strong bond between implants and bone tissues.

As used herein, “calcium deficient apatite” shall mean a group of phosphate minerals, usually referring to hydroxylapatite, fluorapatite and chlorapatite, with high concentrations of OH−, F− and Cl− ions.

As used herein, “capillary” shall mean any of the fine branching blood vessels that form a network between arterioles (the smallest division of an artery) and venules (the smallest division of a vein).

As used herein, “cartilage” shall mean a tough, semitransparent, elastic, flexible connective tissue consisting of cartilage cells scattered through a glycoprotein material that is strengthened by collagen fibers. Examples of cartilage include, but are not limited to, such connective tissue at the joints, the rib cage, the ear, the nose, the throat, between vertebral disks, etc.

As used herein, “cartilage graft” shall mean the transplanted cartilage to correct a defect.

As used herein, “cell adhesion protein” shall mean proteins that hold cells together, and hold them to their substrates.

As used herein, “ceramic” shall mean material that is an inorganic, non-metallic, often crystalline oxide, nitride or carbide material. Ceramic material can include one or two or more metallic and non-metallic elements, with ionic bonds, covalent bonds, or combination of both bonds formed between the elements. Properties of ceramic materials include high melting points, high hardness, excellent wear, with good resistance to degradation in corrosive environment. Ceramic material is non-toxic thus making them biocompatible with cells and tissue and are fully oxides and chemically stable.

As used herein, “chondrocyte” shall mean a differentiated cell responsible for secretion of extracellular matrix of cartilage.

As used herein, “chondrocyte proliferation” shall mean a reparative state that stimulates keratinocytes, fibroblasts, and angiogenesis to promote tissue regeneration by cell division, resulting in the expansion of their population to permit cartilage grafts to integrate with host cartilage in vivo.

As used herein, “chondrocyte repopulation” shall mean the multiplication or reproduction of chondrocytes by cell division, resulting in the expansion of the chondrocyte population to permit cartilage grafts to integrate with host cartilage in vivo.

As used herein, “clamp” shall mean a device which statically compresses the soft tissue graft. The clamp can be made of metal, ceramic, polymers, composites thereof, other material that can compress a soft tissue graft, etc. The material can be porous, permeable, or degradable.

As used herein, “collagen” shall mean the main structural protein found in skin and other connective tissues in the human body. Collagen typically consists of amino acids wound together to form triple-helices of elongated fibrils.

As used herein “collagen production” shall mean the production of collagen in the host cartilage to support cartilage grafts and to permit the cartilage to integrate with the host cartilage in vivo.

As used herein, “damaged soft tissue” shall mean damage of muscles, ligaments and tendons throughout the body. Damaged soft tissue can include injuries such as a sprain, strain, a one off blow resulting in a contusion or overuse of a particular part of the body.

As used herein, “effective amount” and/or “sufficient concentration” shall mean a level, concentration, combination or ratio of one or more components added to the fibrous polymeric scaffolds which promotes differentiation of stem cells to a selected cell type and/or enhances proliferation of desired cells.

As used herein, “electroactivity” shall mean exhibiting electrical activity or responsive to electrical stimuli.

As used herein “electrospun” shall mean produced by the fiber fabrication method called electrospinning.

As used herein, “electrostatic forces” shall mean a force (attractive or repulsive) that exist between electrically charged particle or objects where the attractive or repulsive forces between particles that are caused by their electric charges. In an embodiment, the electric charge is applied to the needle attached to the syringe.

As used herein, “embedded” shall mean fixed firmly and deeply in a surrounding mass, such as implanted. For example, particles affixed to the electrospun fiber. Particles may be affixed on the surface of the fiber or may be disposed in the fiber.

As used herein, “encase” shall mean enclose or cover a cartilage graft or close-fitting around a cartilage graft.

As used herein, “fibers” shall mean a subset of man-made fibers, which are based on synthetic chemicals or natural polymers, such as collagen, silk, and cellulose. Such fibers can be manufactured using techniques such as electrospinning.

As used herein, “fibronectin” shall mean any of a group of glycoproteins of cell surfaces, blood plasma, and connective tissue that promote cellular adhesion and migration.

As used herein, “fibroblast” shall mean a cell of connective tissue, mesodermally derived, that secretes proteins and molecular collagen including fibrillar procollagen, fibronectin and collagenase, from which an extracellular fibrillar matrix of connective tissue may be formed.

As used herein, “fibrous polymeric scaffold” shall mean a scaffold of fibers made from polymers.

As used herein, “fully synthetic” matrices mean that the matrices are composed of man-made material, such as synthetic polymer, or a polymer-ceramic composite, but it does not preclude further treatment with material of biological or natural origin, such as seeding with appropriate cell types, (e.g., seeding with osteoblasts, osteoblast-like cells, and/or stem cells), or treating with a medicament, (e.g., anti-infectives, antibiotics, bisphosphonate, hormones, analgesics, anti-inflammatory agents, growth factors, angiogenic factors, chemotherapeutic agents, anti-rejection agents, and RGD peptides).

As used herein, “functional” shall mean affecting physiological or psychological functions but not organic structure.

As used herein, “glycosaminoglycan” or “GAG” shall mean long linear (unbranched) polysaccharides which consist of repeating disaccharide (double sugar) units and are highly polar and attract water, and typically are useful to the body as a lubricant or as a shock absorber.

As used herein, “glycosaminoglycan deposition” shall mean the increase of glycosaminoglycan in and around the host cartilage which supports capillary growth, fibronectin, and collagen formation at the site of injury so that vascular density of the wound can return to normal.

As used herein, “graft” shall mean the device to be implanted during medical grafting, which is a surgical procedure to transplant tissue without a blood supply, including but not limited to soft tissue graft, synthetic grafts, and the like. The graft can be an allograft or an autograft. An “allograft” is tissue taken from one person for transplantation into another. Allografts can include, most commonly, Achilles and tibialis, patellar and quadricep's tendons. An “autograft” or “autologous graft” is a graft comprising tissue taken from the same subject to receive the graft. Graft can also be allogeneic or xenogenic. In one aspect of an embodiment, the graft is a soft tissue graft. In another embodiment, the soft tissue graft is a tendon. In another embodiment, the graft is a graft for a ligament in a subject, including the ACL. In another embodiment, the tendon graft can be a bone-patellar tendon-bone (BPTB) graft, a semitendinosus or a hamstring-tendon (HST) graft.

As used herein, “graphite” shall mean a stable and naturally occurring allotropes of carbon in metamorphic rock. The natural graphite can be in the form of amorphous graphite, flake graphite or crystalline graphite. Such graphite includes carbons atoms being arranged hexagonally in a planar ring system, with a plurality of layers of the planar ring system being disposed on top of each other, thereby forming graphite having a lattice structure of plural layers. Such lattice structure permits the planar ring system to have a strong bond between carbon atoms in the planar ring system while a weaker bond between carbon atoms in different layers, thus allowing the graphite to have anisotropic electronic, acoustic and thermal properties.

As used herein “graphite particles” shall mean particles of graphite with the size of the particles in the range of nanoscale to microscale.

As used herein, “graft collar” shall mean a device embodying a graft and configured like a collar, that is, having a hollow cylindrical body in a longitudinal direction. A graft collar can be permeable, so the tissue can survive.

As used herein, “graft fixation device” shall mean a device that is useful for affixing a tissue graft to a bone or other body surface, including but not limited to staples, interference (screws with or without washers), press fit EndoButton® devices and Mitek® Anchor devices.

As used herein, “growth factors” shall mean proteins that regulate many aspects of cellular function, including survival, proliferation, migration and differentiation. Examples of growth factors can include cytokines, therapeutic peptides/proteins to aid in bone or dental repair or regeneration, and hormones that bind to specific receptors on the surface of their target cells.

As used herein, “host cartilage” shall mean cartilage of the subject which is being integrated with a cartilage graft from the fibrous polymeric scaffold including graphite particles.

As used herein, “hydroxyapatite” shall mean a particulate calcium phosphate ceramic.

As used herein, “implantable device” according to one embodiment is a surgically appropriate, (e.g., biocompatible), the apparatus having the design and physical properties set forth in more detail below. Preferably, the implantable device is designed and dimensioned to function in the surgical repair, augmentation, or replacement of damaged soft tissue, such as, (e.g., a rotator cuff, including fixation of tendon-to-bone). More particularly, the implantable device comprises a “fibrous, polymeric matrices”.

As used herein, “implantable” or “suitable for implantation” means surgically appropriate for insertion into the body of a host, (e.g., biocompatible), or having the design and physical properties set forth in more detail below.

As used herein, “interference screw” shall mean a type of graft fixation device which anchors a flexible transplant like a tendon or a ligament in an opening in a bone. The screw generally has a screw body, a head at one end of said screw body and a penetrating end at an opposite end of said screw body. The device may be used in, for example, anterior cruciate ligament surgery. The device may be metallic or bioabsorbable and may include, but is not limited to, titanium cannulated interference screws, Poly-L-Lactide (PLLA) interference screws, etc.

As used herein. “laminin” shall mean high-molecular weight proteins of extracellular matrix which enhance cell attachment to the scaffold.

As used herein, “lyophilized”, in regard to a graft collar, shall mean a graft collar that has been rapidly frozen and dehydrated.

As used herein, “matrix” shall mean a three-dimensional structure fabricated from biomaterials. The biomaterials can be biologically-derived or synthetic.

As used herein, “mechano-transducer” shall mean that the host cartilage generates a measurable response to mechanical stimulation and is able to convert the energy from one form into another.

As used herein, “mesh” means a network of material. In one embodiment, the mesh may be woven synthetic fibers, non-woven synthetic fibers, microfibers and nanofibers suitable for implantation into a mammal, (e.g., a human). The woven and non-woven fibers may be made according to well known techniques. The microfiber or nanofiber mesh may be made according to techniques known in the art and those disclosed in, (e.g., International application no. PCT/US2008/001889 filed on Feb. 12, 2008 to Lu et al.), which application is incorporated by reference as if recited in full herein. Fibers of the mesh may be aligned or unaligned.

As used herein, “micrometer-scale” or “micron” shall mean a metric unit of length equal to 0.001 mm, or about 0.000039 inch having a symbol μm.

As used herein, “multiphase scaffold” shall mean that the scaffold includes more than one phase of the scaffold. The scaffold may have more than one phase, depending on the anatomical architecture of the ligament or tendon to be repaired, fixed, augmented, or replaced. An exemplary number of phases is from about 1 to about n, such as for example, from about 2 to about 4, preferably 3 or 4. As noted above, in such multiphasic embodiments, each phase of the scaffold is continuous from phase-to-phase. In one embodiment, such as in the present fully synthetic implantable multiphase scaffolds, the interface between one phase and the next is designed, e.g., by sintering and other means described in more detail below, to mimic the natural anatomical structure, e.g., of a tendon or of a ligament, particularly the insertion sites thereof.

As used herein, “musculoskeletal cell” shall mean a chondrocyte, fibrochondrocyte, fibroblast or osteoblast.

As used herein, “nanofiber mesh” shall mean a flexible netting of nanofibers, oriented such that at least some of the nanofibers are not parallel to others of the nanofibers.

As used herein, “nanofiber scaffold” is constructed of “nanofibers.”

As used herein, “nanofiber” shall mean fibers with diameters no more than 1000 nanometers. In one embodiment, a “nanofiber” is a biodegradable polymer that is electrospun into a fibrous polymeric scaffold as described in more detail herein below. The nanofibers of the fibrous polymeric scaffold are oriented in such a way (i.e., aligned or unaligned) so as to mimic the natural architecture of the soft tissue to be repaired. Moreover, the nanofibers and the subsequently formed fibrous polymeric scaffolds are controlled with respect to their physical properties, such as for example, fiber diameter, pore diameter, and porosity so that the mechanical properties of the fibrous polymeric scaffolds are similar to the native tissue to be repaired, augmented or replaced. Thus, in the case of a rotator cuff repair, the fibrous polymeric scaffolds are able to regenerate the native insertion of tendon-to-bone through interface tissue engineering and promote tendon-to-bone integration and biological fixation. In another embodiment, such a fibrous polymeric scaffold may be multiphasic, such as (e.g., biphasic). One aspect of such multiphasic fibrous polymeric scaffold is that each phase is “continuous” with the phase adjacent to it. Thus, in the present fibrous polymeric scaffold, the interface between one phase and the next is designed, (e.g., by electrospinning and other means described in more detail below), to mimic the natural anatomical transition between, (e.g., tendon and bone at a tendon-to-bone interface). By designing the fibrous polymeric scaffold of the embodiment so that the phases are continuous, improved fixation and function is achieved by minimizing stress concentrations and mediating load transfer between tendon and bone compared to prior systems. In another embodiment, the fibrous polymeric scaffold may be engineered to remain in place for as long as the treating physician deems necessary. Typically, the fibrous polymeric scaffold will be engineered to have biodegraded between 6-18 months after implantation, such as for example 12 months.

As used herein, “nanometer-scale” shall mean size of a structure that is in a range of approximately one nanometer to one micrometer.

As used herein, “negatively charged” shall mean having a negative charge; “electrons are negative” electronegative, negative. charged − of a particle or body or system; having a net amount of negative electric charge.

As used herein, “osteoblast” shall mean a bone-forming cell that is derived from mesenchymal osteoprogenitor cells and forms an osseous matrix in which it becomes enclosed as an osteocyte. The term is also used broadly to encompass osteoblast-like, and related, cells, such as osteocytes and osteoclasts. An “osteoblast-like cell” means a cell that shares certain characteristics with an osteoblast (such as expression of certain proteins unique to bones) but is not an osteoblast. “Osteoblast-like cells” include preosteoblasts and osteoprogenitor cells. Preferably the cells are from a compatible human donor. More preferably, the cells are from the patient (i.e., autologous cells).

As used herein, “osteointegrative” shall mean ability to chemically bond to bone.

As used herein, “particle” shall mean a small localized object to which can be ascribed several physical or chemical properties such as volume, density or mass.

As used herein, “particle reinforcement” means a process for forming a composite with a higher strength than the original material (e.g., a polymer) by adding particles of a reinforcing material with a higher strength (e.g., a ceramic).

As used herein, “PDGF” shall mean platelet-derived growth factor.

As used herein, “phase” shall mean a continuous construct of material of matrices. Such matrices are in a solid form. Such phase of the matrices is in solid form. The matrices can include one phase which comprises material that is uniformly distributed throughout the matrices. Further, the matrices can include more than one phase.

As used herein, “PGA” shall mean polyglycolide or poly(glycolic) acid.

As used herein, “PLA” shall mean poly(lactic) acid or polylactic acid or polylactide.

As used herein, “PLGA” shall mean poly(lactic-co-glycolic) acid, a biodegradable and biocompatible copolymer.

As used herein, “PCL” shall mean polycaprolactone, a biodegradable polyester with a low melting point of around 60° C. and a glass transition temperature of about −60° C. with a chemical formula (C₆H₁₀O₂)ₙ, where n stands for any number.

As used herein, “polymer” means a chemical compound or mixture of compounds formed by polymerization and including repeating structural units. Polymers may be constructed in multiple forms and compositions or combinations of compositions.

As used herein, “polymer blend” shall mean a mixture of two or more polymers of varying concentrations.

As used herein, “polymer solution” shall mean as solution containing polymers mixed therein.

As used herein, “polymeric blend” shall mean a mixture of two or more polymers of varying concentrations.

As used herein, “polymeric fibers” shall mean a subset of man-made fibers, which are based on a polymer.

As used herein, “polymeric matrices” shall mean matrices produced from fibrous polymer.

As used herein, “porosity” shall mean the ratio of the volume of interstices of a material to a volume of a mass of the material.

As used herein, “proteoglycan” shall mean a compound consisting of a protein bonded to glycosaminoglycan groups, present especially in connective tissue.

The “rotator cuff” refers to the group of muscles and tendons that surround the humeral head. Specifically, the rotator cuff consists of a group of four muscles and tendons, including the supraspinatus, infraspinatus, teres minor, and subscapularis, which function in synchrony to stabilize the glenohumeral joint as well as to actively control shoulder kinematics. The supraspinatus tendon inserts into the humeral head via a direct insertion exhibiting region-dependent matrix heterogeneity and mineral content.

As used herein, “scaffold” shall mean an arrangement of fibers that can support growth, maintenance and differentiation of one or more tissue and cell types.

As used herein, “sinter” or “sintering” shall mean densification of a particulate polymer compact involving a removal of pores between particles (which may be accompanied by equivalent shrinkage) combined with coalescence and strong bonding between adjacent particles. The particles may include particles of varying size and composition, or a combination of sizes and compositions. For example, sintering a polymer would involve heating the polymer above the glass transition temperature, wherein the polymer chains rearrange and link together to form sintering necks.

As used herein, “soft tissue graft” shall mean a graft which is not synthetic, and can include autologous grafts, syngeneic grafts, allogeneic grafts, and xenogeneic graft.

As used herein, “soft tissue” includes, tendon and ligament, as well as the bone to which such structures may be attached. Preferably, “soft tissue” refers to tendon- or ligament-bone insertion sites requiring surgical repair, such as for example tendon-to-bone fixation.

As used herein, “sonicating” shall mean to subject to ultrasonic vibration so as to fragment the cells.

As used herein, “stem cell” means any unspecialized cell that has the potential to develop into many different cell types in the body. such as mesenchymal osteoprogenitor cells, osteoblasts, osteocytes, osteoclasts, chondrocytes, and chondrocyte progenitor cells. Preferably the cells are from a compatible human donor. More preferably, the cells are from the patient (i.e., autologous cells).

As used herein, “synthetic” shall mean that the material is not of a human or animal origin.

As used herein, “synthetic graft material”, i.e., first phase, means man-made material that is intended for insertion into a host body. The synthetic graft material used in the first phase may be made from aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, degradable polyurethanes, or biopolymers, or a blend of two or more of the preceding polymers. Preferably, the synthetic graft material used in the first phase is poly(lactide-co-glycolide), poly(lactide) or poly(glycolide).

As used herein, “unaligned fibers” shall mean groups of fibers which are randomly or pseudo-randomly oriented.

As used herein, “xenogenic”, shall mean from a different species. As applied to grafts, xenogenic shall mean that the graft is derived from a material originating from a species other than that of the subject receiving the graft.

As used herein, “zone of chondrocyte death” or “ZoCD” shall mean a dense region of necrosed cells that lines the periphery of the wound edge in cartilage autografts and damaged host tissue, creating a barrier for chondrocytes to migrate to the graft periphery, thus limiting cartilage-to-cartilage healing.

As used herein, all numerical ranges provided are intended to expressly include at least the endpoints and all numbers that fall between the endpoints of ranges.

### EMBODIMENTS

The following detailed description of embodiments and examples are set forth to aid in an understanding of the subject matter of this disclosure but are not intended to, and should not be construed to, limit in any way the disclosure as a whole.

Each embodiment disclosed herein is contemplated as being applicable to each of the other disclosed embodiments. Thus, all combinations of the various elements described herein are within the scope of this disclosure.

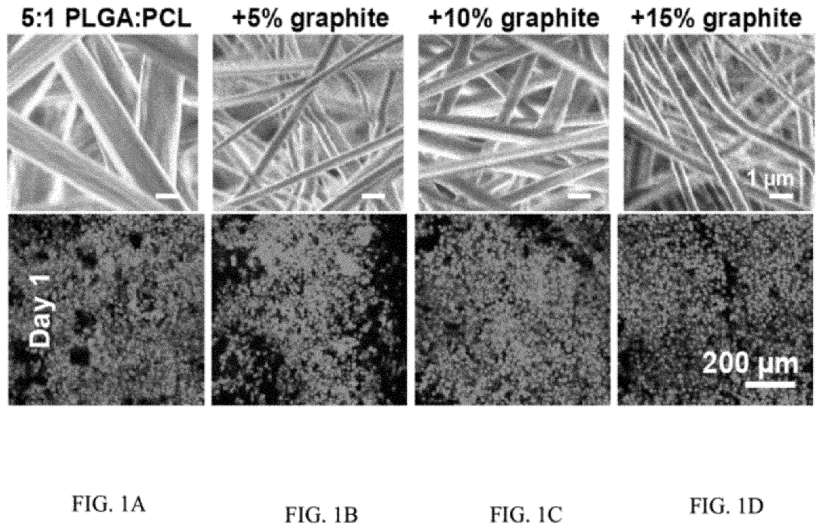

This application describes in one embodiment a fibrous polymeric scaffold comprising electrospun fibers including a polymeric blend of poly(lactic-co-glycolic) acid (PLGA) and poly(ε-caprolactone) (PCL), as shown in FIG. 1A.

In another embodiment, a fibrous polymeric scaffold comprising electrospun fibers including a polymeric blend of poly(lactic-co-glycolic) acid (PLGA) and poly(ε-caprolactone) (PCL), in addition to 5% graphite particles embedded in the fibers, as shown FIG. 1B.

In another embodiment, a fibrous polymeric scaffold comprising electrospun fibers including a polymeric blend of poly(lactic-co-glycolic) acid (PLGA) and poly(ε-caprolactone) (PCL), in addition to 10% graphite particles embedded in the fibers, as shown FIG. 1C.

In yet another embodiment, a fibrous polymeric scaffold comprising electrospun fibers including a polymeric blend of poly(lactic-co-glycolic) acid (PLGA) and poly(ε-caprolactone) (PCL), in addition to 15% graphite particles embedded in the fibers, as shown FIG. 1D.

Such a fibrous polymeric scaffold comprising electrospun fibers including polymeric blend and graphite enhances chondrocyte proliferation and glycosaminoglycan deposition, which facilitates cartilage graft integration with host cartilage.

The fibrous polymeric scaffold containing can also provide cell adhesion and matrix deposition in musculoskeletal tissue engineering.

Such fibrous polymeric scaffolds are formed for example, an electrospinning apparatus by sonicating the graphite particles in acetic acid for 72 hours, adding PLGA and PCL to the solution (32% w/w), vortexing until the polymers are solubilized, and electrospinning using (10 kV, 15 cm, 0.75 mL/hr, 40% RH) the solution onto a stationary collecting plate.

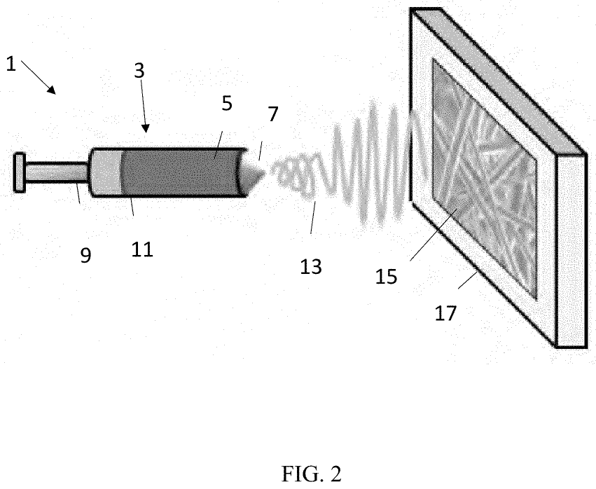

Such electrospinning apparatus is exemplified by way of FIG. 2. FIG. 2 shows an electrospinning apparatus 1 including a syringe 3 configured with a vessel 11 and connected at one end of the vessel 11 to a needle 7 in the form of a cone from which a jet of solution 13 ejects toward the grounded plate 17. A syringe plunger 7 connects to the vessel 11 at the other end of the syringe 3, and applies pressure on the content of the cylindrical vessel, in particular the content in the cylindrical vessel is the polymer solution 5. The polymer solution 5 ejects towards the grounded plate 17 and forms a batch of polymeric fibers 15 on the grounded plate 17. The syringe 3 is configured with a volume of 5 ml, the needle 7 connected to cylindrical vessel of the syringe at one end is configured with a 26G needle. The syringe plunger that provides pressure on the content of the cylindrical vessel, ejects the content at a flow rate in the range of 0.4-1.0 ml/hr. The electrospinning apparatus is in an enclosing cabinet, which includes a humidifier that maintains the humidity in the enclosing cabinet in a range of 45% to 55%.

Electrospinning, short for electrostatic spinning, involves the fabrication of fibers by applying a high electric potential to a polymer solution. The material to be electrospun, is loaded into a syringe or spoon, and a high potential is applied between the solution and a grounded substrate. As the potential increases, the electrostatic force applied to the polymer solution overcomes surface tension, distorting the solution droplet into a Taylor cone from which a jet of solution ejects toward the grounded plate or a cylindrical vessel. The jet splays into randomly oriented fibers. These fibers have diameters ranging from nanometer scale to greater than 1 μm and are deposited onto the grounded plate or onto objects inserted into the electric field forming a non-woven batch of polymeric fibers.

Further, a distance between the tip of the needle and the grounded plate during the electrospinning of the batch of polymeric fibers, is in a range of 10-15 cm. A voltage applied to the needle during the electrospinning of the batch of polymeric fibers is in the range of 8-12 kV.

Resulting fibers are smooth, uniform, and unaligned, with nanometer-scale diameters.

In one aspect of this embodiment, the graphite particles embedded in the fibrous polymeric scaffold impart electroactivity to the scaffold, which mimics the native nature of cartilage. Cartilage is a natural mechanotransducer, composed of a charged extracellular matrix phase, a liquid phase (water), and an ion phase. Accordingly, the mechanical forces move the fluid and ion phases through the matrix, a streaming potential is generated which directs cell response.

Such fibrous polymeric scaffold containing graphite has been shown according to in vitro studies to demonstrate that the fibers enhances chondrocyte proliferation, glycosaminoglycan deposition, and collagen production in a dose-dependent manner, all of which are necessary to integrate cartilage grafts with host cartilage in vivo. Further, the fibrous polymeric scaffold containing graphite may also attract negatively charged cell adhesion proteins such as fibronectin and laminin, which will enhance cell attachment to the scaffold. Improved cell adhesion to the fibrous polymeric scaffold containing graphite, teamed with strategic biomimicry of cartilage electroactivity, may facilitate chondrocyte repopulation of the ZoCD and subsequent matrix deposition by chondrocytes to integrate cartilage grafts with host cartilage.

In another aspect of this embodiment, the electrospun fibers of fibrous polymeric scaffold encase a cartilage graft, to support integration of the cartilage graft with host cartilage.

A further aspect of this embodiment, the graphite particles in the fibrous polymeric scaffold impart electroactivity to the fibrous polymeric scaffold to biomimic native properties of the host cartilage operating as a mechano-transduces.

In another embodiment of this disclosure, the fibrous polymeric scaffold including the graphite particles embedded in the electrospun fibers of the fibrous polymeric scaffold attract cell adhesion proteins from the host cartilage.

In another embodiment of this disclosure, the fibrous polymeric scaffold including the graphite particles embedded in the electrospun fibers of the fibrous polymeric scaffold attract fibronectin and laminin from the host cartilage.

In another embodiment of this disclosure, the fibrous polymeric scaffold including the graphite particles embedded in the electrospun fibers enhance chondrocyte proliferation in the host cartilage.

In another aspect of this embodiment, the host cartilage includes a zone of chondrocyte death (ZoCD), and the graphite particles embedded in the electrospun fibers of the fibrous polymeric scaffold facilitate chondrocyte repopulation in the ZoCD of the host cartilage.

In yet another aspect of this embodiment, the fibrous polymeric scaffold including the graphite particles embedded in the electrospun fibers enhance glycosaminoglycan deposition in the host cartilage.

In another aspect of this embodiment of this disclosure, the fibrous polymeric scaffold including the graphite particles embedded in the electrospun fibers enhance collagen production in the host cartilage.

In another embodiment of this disclosure, the fibrous polymeric scaffold including the graphite particles embedded in the electrospun fibers constitute 5% to 15% by weight in the electrospun fibers.

In another aspect of this embodiment, the fibrous polymeric scaffold including the graphite particles embedded in the electrospun fibers enhance cell attachment to the fibrous polymeric scaffold.

In another aspect of this embodiment, the fibrous polymeric scaffold including the graphite particles embedded in the electrospun fibers have an unaligned arrangement in the fibrous polymeric scaffold.

In another aspect of this embodiment, the fibrous polymeric scaffold including the graphite particles embedded in the electrospun fibers, at least some of the electrospun fibers having the graphite particles embedded therein have a nanometer-scale diameter.

In this disclosure, a method is described for preparing a fibrous polymeric scaffold to promote the chondrocyte proliferation and viability. A method, according to one embodiment, includes (a) dissolving one or more polymers, individually or as a polymer blend, including graphite particles in a solution, to form a polymer solution, (b) performing electrospinning using the polymer solution in which the one or more polymers including the graphite particles, to form one or more batches of fibrous polymeric nanofibers, and (c) forming one or more fibrous, polymeric scaffolds using the one or more batches of fibrous polymeric nanofibers including the graphite particles in which the one or more polymers is dissolved in (a).

An another aspect of such an embodiment includes the method for generating a fibrous polymeric scaffold, the method comprising: (a) dissolving poly(lactic-co-glycolic) acid (PLGA) and poly(ε-caprolactone) (PCL), individually or as a polymer blend, in a Class 3 solvent, to form a polymer solution (Step S101); (b) adding graphite particles (Step S103) to the polymer solution (Step S105); (c) performing electrospinning using the polymer solution in which the graphite particles have been added (Step S107), to form polymeric fibers, the graphite particles constituting 5% to 15% by weight in the polymer solution employed in the electrospinning; and (d) encasing a cartilage graft with the electrospun fibers as the fibrous polymeric scaffold (Step S109), to support integration of the cartilage graft with host cartilage.

In this embodiment, the polymer dissolved in the solvent can be a polymer blend of poly(lactic-co-glycolic) acid (PLGA) and poly(ε-caprolactone) (PCL). Preferably, the solvent can include acetic acid. Further, the acetic acid can include a glacial acetic acid. In step (a) (Step S101), a mixture of the solvent with the polymer undergo vortexing, to form a polymer solution. The vortexting of the polymer solution can last for 3 to 4 hours to dissolve the polymers in the solution. After the polymer solution has been vortexed, the solution can include a concentration of polymer up to 33% (w/w). The solution is loaded into an electrospinning apparatus where it undergoes green electrospinning to form one or more batches of fibrous polymeric nanofibers including graphite particles embedded therein (Step S107). Preferably, during the electrospinning step, the ejection flow rate of polymer solution from the syringe is in a range of 0.4-1 ml/hr. Subsequent, to this, the one or more polymeric batches can be used for forming fibrous polymeric scaffolds, including the graphite particles.

Additional steps may optionally be added to the method to impart additional features or characteristics. For example, the method may include adding additional components to the fibrous, polymeric scaffolds, to form a mesh, graft collar, implantable device, multiphase scaffold, etc. The additional components include active agents, one or more growth factors, hydroxyapatite or a calcium phosphate, calcium-deficient apatite (CDA). Such additional components permit the mesh, graft collar, implantable device, or multiphase scaffold, to provide a functional interface between multiple tissue types. Such components can further include an active pharmaceutical ingredient such as, but not limited to, an anti-inflammatory, an antibiotic or a pain medicament added to the fibrous, polymeric scaffolds to enhance treatment and/or healing of the subject upon implantation of the mesh, graft collar, implantable device, or multiphase scaffold.

The method may further include seeding the fibrous, polymeric scaffolds with selected musculoskeletal cells or stem cells which differentiate into the musculoskeletal cells, or with soft tissue graft to replaces or repairs damaged soft tissue.

Examples of musculoskeletal cells which can be seeded onto these fibrous, polymeric matrices include chondrocytes, fibro chondrocytes, fibroblasts and osteoblasts. In some embodiments fibrous, polymeric matrices may be seeded with a single type of musculoskeletal cell, a mixture of selected musculoskeletal cells and/or stem cells which differentiate into a selected musculoskeletal cell or mixture of selected musculoskeletal cells. For multiphasic fibrous, polymeric matrices, the first phase and the one or more additional phases may be seeded with the same selected musculoskeletal cell or mixture of cells, and/or stem cells which differentiate into the selected musculoskeletal cell or mixture of cells. Alternatively, the first phase and the one or more additional phases may be seeded with different selected musculoskeletal cells.

According to one embodiment, the method includes the graphite particles embedded in the electrospun fibers of the fibrous polymeric scaffold which impart electroactivity to the fibrous polymeric scaffold to biomimic native properties of the host cartilage operating as a mechano-transducer.

Examples of graphite particles which can be embedded in the fibrous polymeric scaffolds include graphite flakes, graphite fibers, graphite crystalline small flakes, amorphous graphite, lump graphite, pyrolytic graphite, etc.

As discussed herein, a method can include steps to prepare a fibrous polymeric scaffold in much safer way to support integration of the cartilage graft with host cartilage.

According to another embodiment the method of preparing the fibrous polymeric scaffolds, the graphite particles embedded in the electrospun fibers of the fibrous polymeric scaffold attract cell adhesion proteins from the host cartilage.

In another embodiment of this disclosure, the method of preparing the fibrous polymeric scaffolds attract fibronectin and laminin from the host cartilage.

In another aspect of this embodiment, the fibrous polymeric scaffold enhances chondrocyte proliferation in the host cartilage.

In yet another aspect of this embodiment, the host cartilage includes a zone of chondrocyte death (ZoCD), and the graphite particles embedded in the electrospun fibers of the fibrous polymeric scaffold facilitate chondrocyte repopulation in the ZoCD of the host cartilage.

In another aspect of the embodiment, the graphite particles embedded in the electrospun fibers of the fibrous polymeric scaffold enhance glycosaminoglycan deposition in the host cartilage.

In another aspect of the embodiment, the graphite particles embedded in the electrospun fibers of the fibrous polymeric scaffold enhance collagen production in the host cartilage.

In another embodiment of this disclosure, the graphite particles embedded in the electrospun fibers of the fibrous polymeric scaffold enhance cell attachment to the fibrous polymeric scaffold.

In another embodiment of this disclosure, the method of preparing the electrospun fibers encasing the cartilage graft forms an unaligned arrangement of the fibrous polymeric nanofibers.

In another embodiment of this disclosure, the method of preparing the electrospun fibers encasing the cartilage graft have a nanometer-scale diameter.

Further non-limiting details are described in the following Experimental Details section which is set forth to aid in an understanding of the invention but is not intended to, and should not be construed to, limit in any way the scope of this disclosure.

### EXPERIMENTAL SECTION

### Introduction:

Articular cartilage is comprised of a negatively charged proteoglycan and collagen matrix, and under physiological loading the induced fluid and ion flow generates a streaming potential that directs chondrocyte response^{1,2}. Given the fixed charged density of cartilage matrix, electroactive scaffolds have emerged as a biomimetic approach to cartilage regeneration^{3,4}. In addition to being conductive, graphite has been shown to be biocompatible⁵ and supports fibroblast growth⁶. Therefore, the objective of this study is to evaluate chondrocyte response to conductive, graphite-doped polymeric fibers tailored for cartilage tissue engineering. It is hypothesized that graphite-doped fibers will enhance chondrocyte proliferation and cartilage-like matrix synthesis.

### Materials and Methods:

### Fabrication:

A 5:1 blend of polylactide-co-glycolide (PLGA, 50/50, MW=106 kDa, Lakeshore) and poly-ε-caprolactone (PCL, Mn≈70,000-90,000, Sigma-Aldrich) was electrospun⁷ with or without graphite (5, 10, and 15% w/w) incorporated by sonicating⁸ and adding to the polymer melt (32% w/v).

### Characterization:

Fibers were characterized (n=3/method) with x-ray diffraction, Raman spectroscopy and thermogravimetric analysis. Conductivity (n=5) was calculated from bulk fiber resistance measured in a custom parallel plate chamber⁹.

### Cell Culture:

Chondrocytes isolated from neonatal bovine femoral cartilage (Green Village Packing Co.) were seeded onto scaffolds at 100,000 cells/cm² in ITS media.

### Cell Growth:

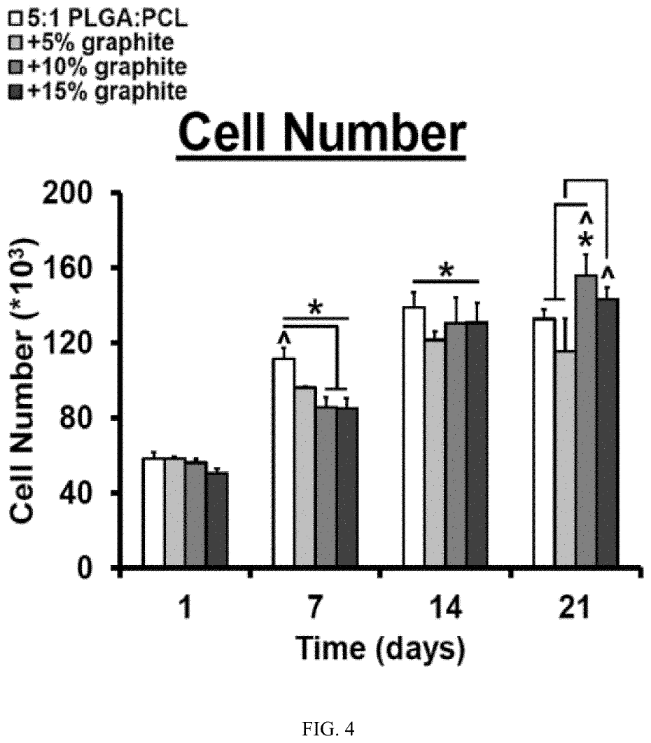

Cell viability (n=2) and proliferation (n=5) were assessed via LIVE/DEAD staining and a Picogreen dsDNA assay, respectively, as shown in FIG. 4.

### Mineralization Potential:

Alkaline phosphatase activity (n=5) was quantified with a calorimetric assay (BioVision).

### Matrix Deposition:

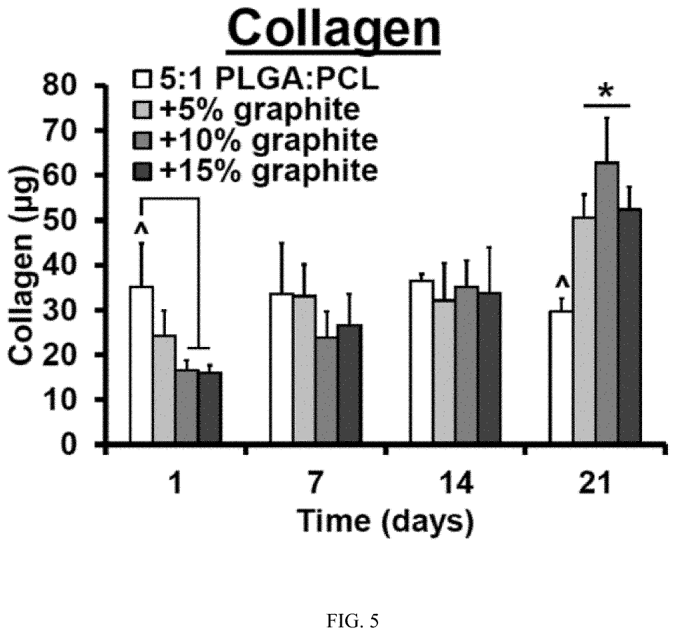

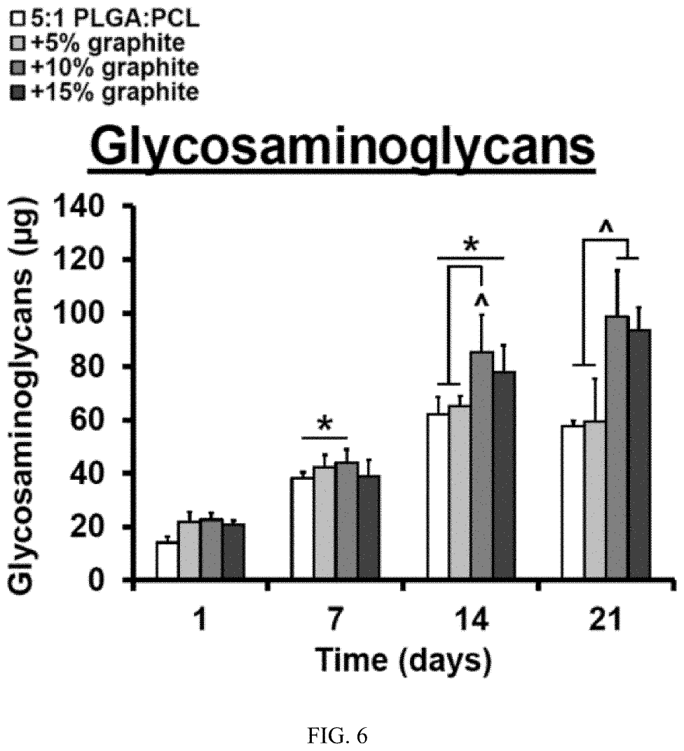

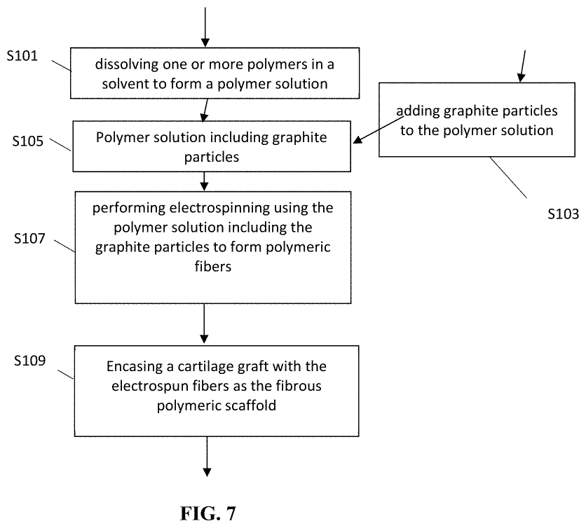

Glycosaminoglycan shown in FIG. 6 and collagen production (n=5), as shown in FIG. 5, were measured with dimethylmethylene blue-binding and hydroxyproline assays, respectively (Sigma-Aldrich).

### Statistical Analysis:

ANOVA and Tukey-Kramer post hoc tests were performed for all pair-wise comparisons (p<0.05).

### Results and Discussion:

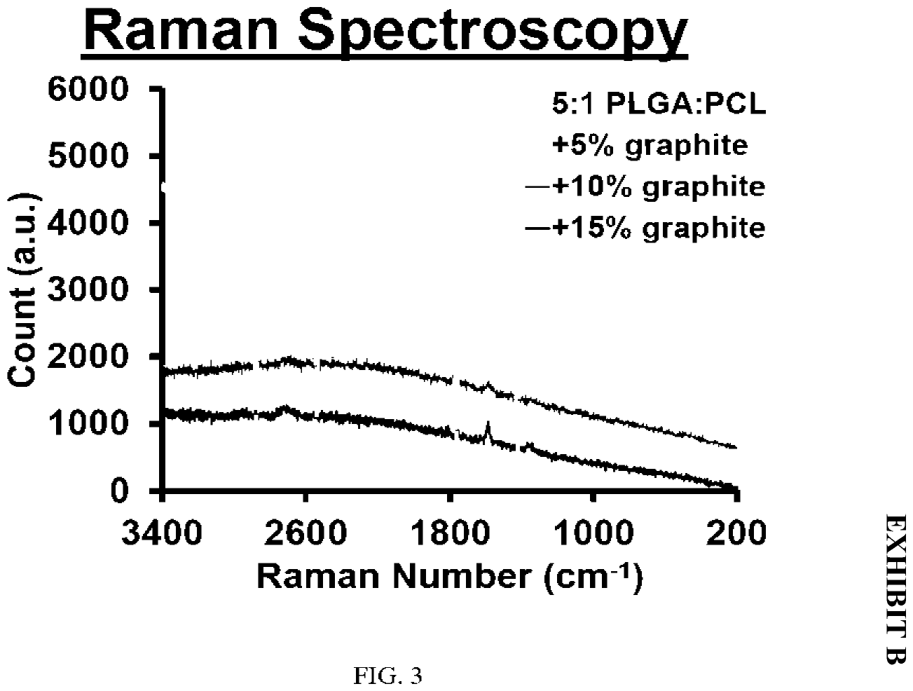

Graphite incorporation was confirmed by XRD, TGA and Raman spectroscopy (FIG. 3), imparting a dose-dependent increase in conductivity (p<0.05) which measured 6.59±0.04, 7.37±0.06, 8.25±0.76, and 9.02±0.08 μS/m for 0, 5, 10, and 15% w/w fibers, respectively. Cells grew over time on all scaffolds, with the highest cell number measured for the 10% and 15% groups (day 21, \hat{ }p<0.05). Both GAG and collagen production significantly increased with graphite incorporation (\hat{ }p<0.05). These observations collectively demonstrate that the electroactive scaffold enhances chondrocyte growth and matrix synthesis. Additionally, the composite scaffolds overcome the limitations of similarly conductive scaffold materials such as polyaniline and polypyrrole, which are non-degradable and more hydrophobic than graphite¹⁰.

### Conclusions:

The results of this study demonstrate that graphite nanoparticles enhance chondrocyte proliferative capacity and cartilaginous matrix synthesis by imparting conductivity to the scaffold. In future studies, the underlying cellular mechanism of graphite-doped scaffold-mediated cartilage regeneration will be explored.

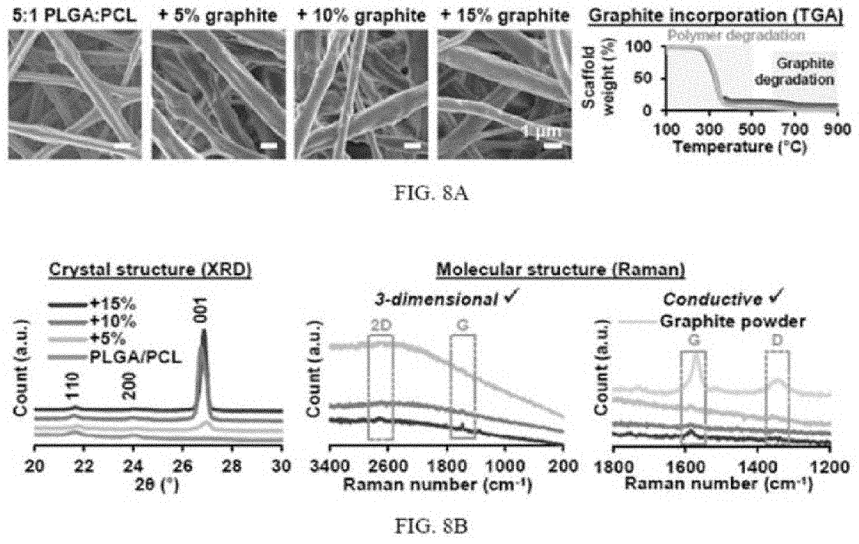

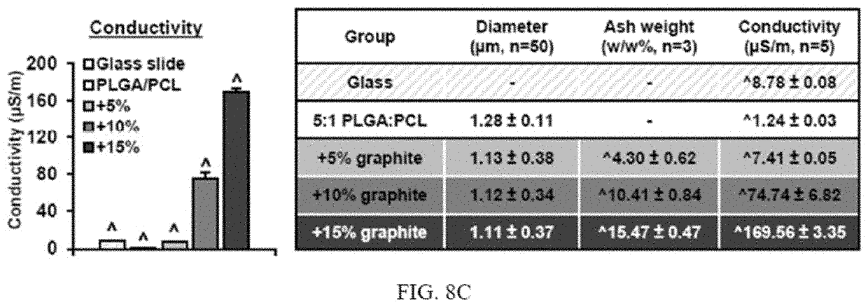

### Characterization of Graphite-Containing Microfibers (FIGS. 8A-8C).

PLGA/PCL fibers containing 0, 5, 10, and 15% w/w graphite were fabricated via electrospinning (FIGS. 8A-8C). Graphite incorporation did not affect the fiber diameter (n=50), and was quantified within 1% w/w of theoretical values with thermogravimetric analysis (TGA, n=3, \hat{ }p<0.05 between groups) (FIG. 8A). The presence of conductive graphite in the fibers was confirmed using x-ray diffraction (XRD, n=3) and Raman spectroscopy (n=3), which also found evidence for electron mobility based on the presence of the D band (1350 cm⁻¹) in raw graphite powder and all graphite-containing fiber groups (FIG. 8B). Fiber conductivity, measured with a custom parallel plate device, increased with increasing graphite dose (n=5, \hat{ }p<0.05) (FIG. 8C).

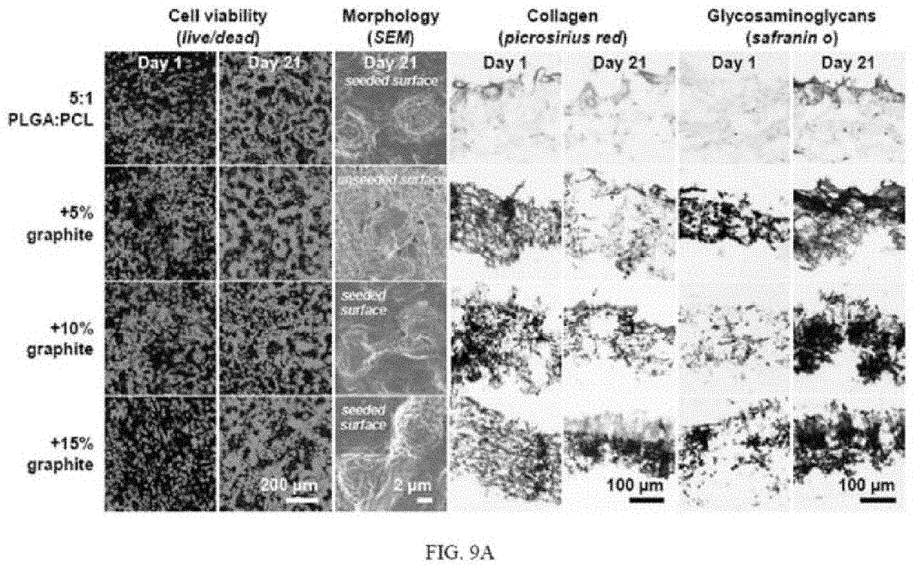

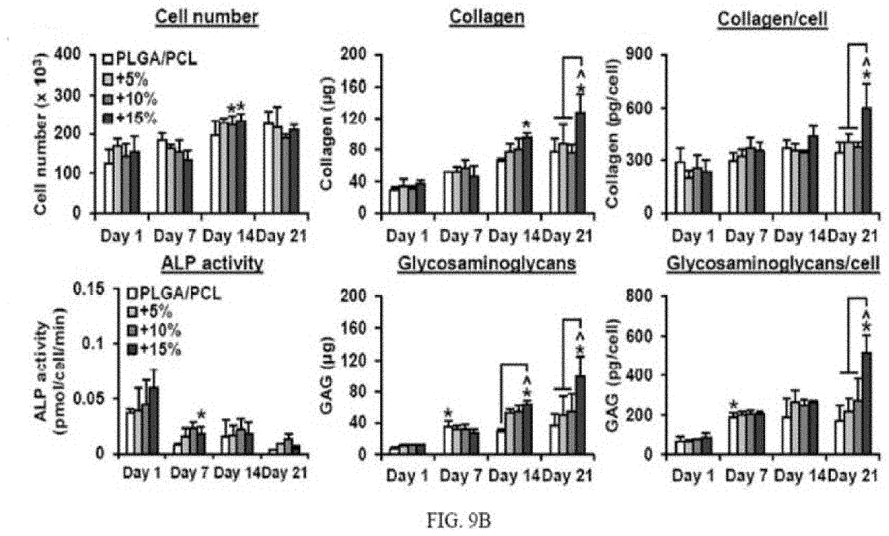

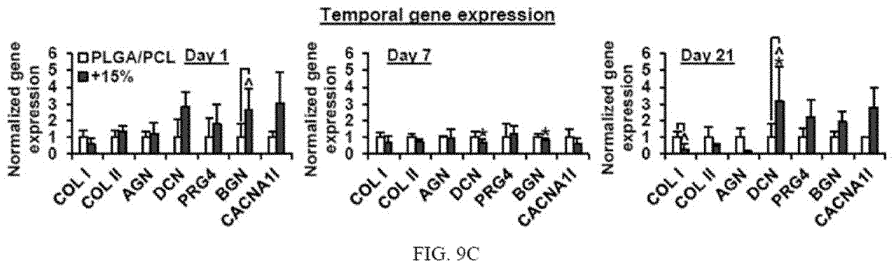

### Cell Response on Graphite Microfibers (FIGS. 9A-9C).

Cell viability (live/dead, n=2) and morphology (SEM, n=2), as well as collagen (picrosirius red, n=3) and glycosaminoglycan (GAG, safranin o, n=3) matrix deposition were visualized (FIG. 9A). Cell number increased over time, with 10 and 15% graphite enhancing proliferation by day 14 (n=5, \hat{ }p<0.05 over time) (FIG. 9B). A collagen and GAG-rich matrix was deposited in all groups, especially the 15% graphite (n=5, \hat{ }p<0.05 between groups) with minimal mineralization potential measured via ALP activity (n=5) (FIG. 9B). Selective proteoglycans regulation and downregulation of collagen type I (n=5, \hat{ }p<0.05) suggest a more hyaline-type cartilage formed in the presence of graphite (FIG. 9C).

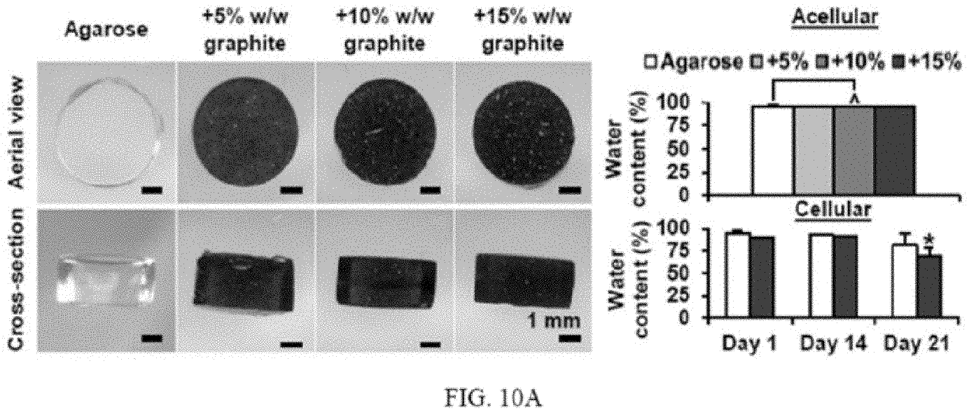

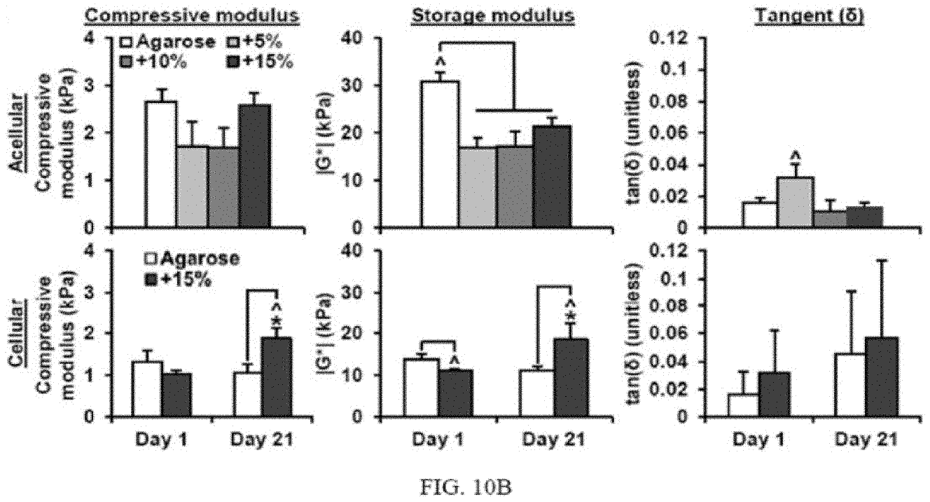

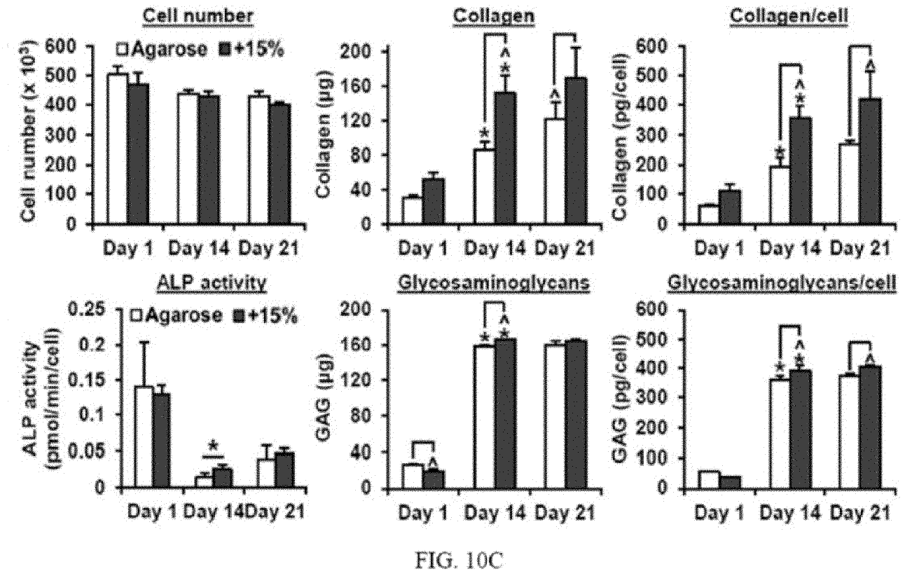

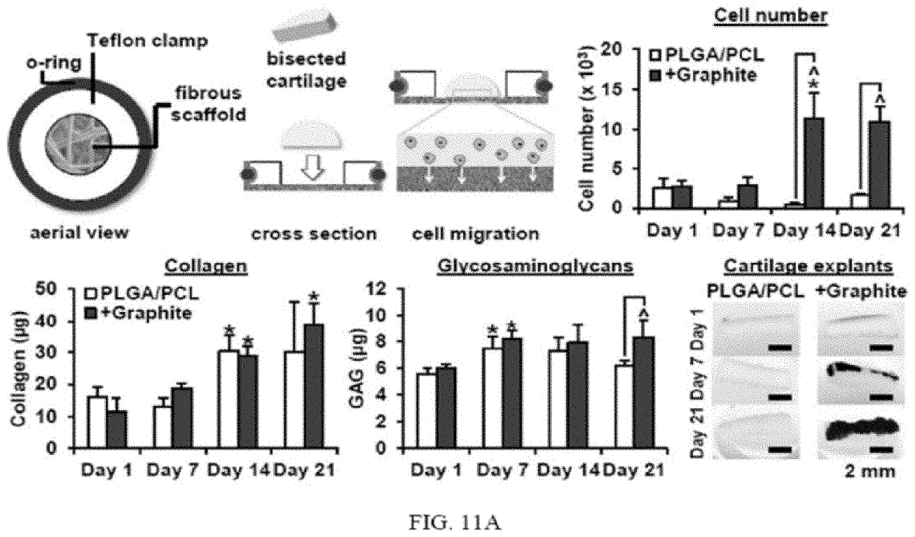

### Graphite Hydrogel Characterization and Cell Response (FIGS. 10A-10C).

Hydrogels were uniform in size and in shape, with graphite distributed throughout the gel (flatbed scanner, n=2) (FIG. 10A). The water content was nearly 100% in all groups, and decreased over time in cellularized graphite gels (n=5, \hat{ }p<0.05 over time) (FIG. 10A). Acellular, graphite-free hydrogels elicited a greater storage modulus than all graphite-containing gels (n=3, \hat{ }p<0.05) (FIG. 10B). When the gels were cellularized, both moduli decreased (FIG. 10B). Graphite enhanced both moduli over time (*p<0.05) and compared to graphite-free gels at day 21 (\hat{ }p<0.05 between groups) (FIG. 10B). The low tan(δ) (<1) in all gels suggests their elastic properties dominate over viscous properties (FIG. 10B). Cells remained viable but did not proliferate throughout culture (n=5) (FIG. 10C). Graphite incorporation enhanced collagen and glycosaminoglycan matrix deposition (n=5, p<0.05) with minimal mineralization potential detected in any group via ALP activity (n=5) (FIG. 10C).

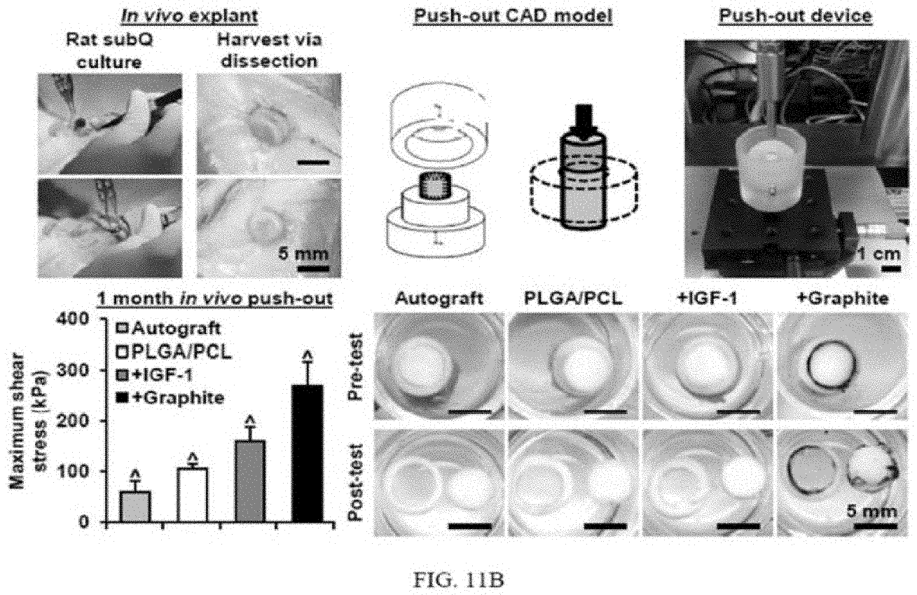

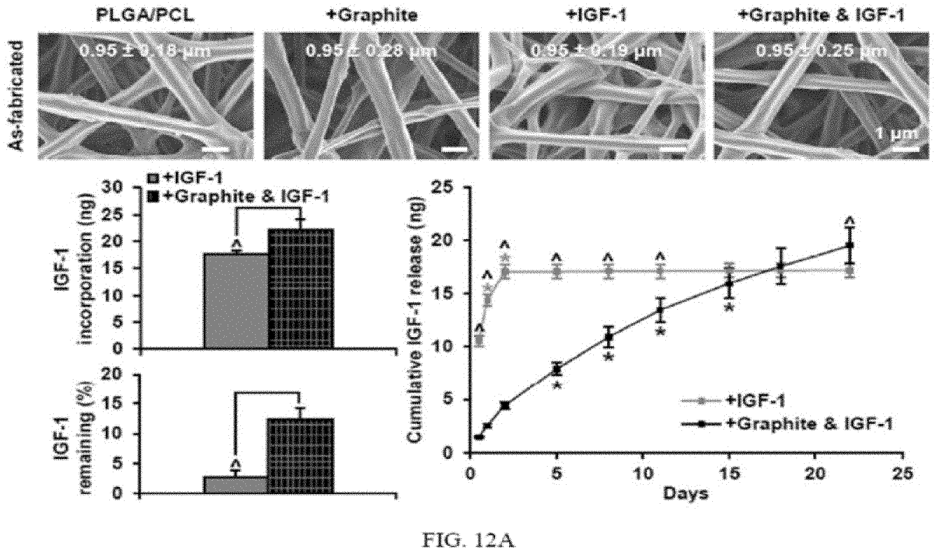

Enhanced Cell Migration and Functional Mechanical Repair with Graphite Microfibers (FIGS. 11A and 11B).

When the cut face of cartilage explants was cultured atop polymer scaffolds, significantly more cells were measured on graphite-containing scaffolds by day 14 (n=5, \hat{ }p<0.05 between groups) (FIG. 11A). Without graphite, cell number did not increase over time (FIG. 11A). Graphite also enhanced glycosaminoglycan deposition by day 21 of culture (n=5, \hat{ }p<0.05) with collagen deposition increasing over time in the graphite group (n=5, *p<0.05 over time) (FIG. 11A). Graphite scaffolds adhered to cartilage explants throughout culture, which confounded quantification of cells and matrix in this group (FIG. 11A). After 1 month of culture in vivo in a rat dorsal subcutaneous model, bovine osteochondral explants repaired with graphite fibers demonstrated improved graft-host cartilage integration compared to autografts, PLGA/PCL fiber repair, and IGF-1 fiber repair (n=8, \hat{ }p<0.05) (FIG. 11B).

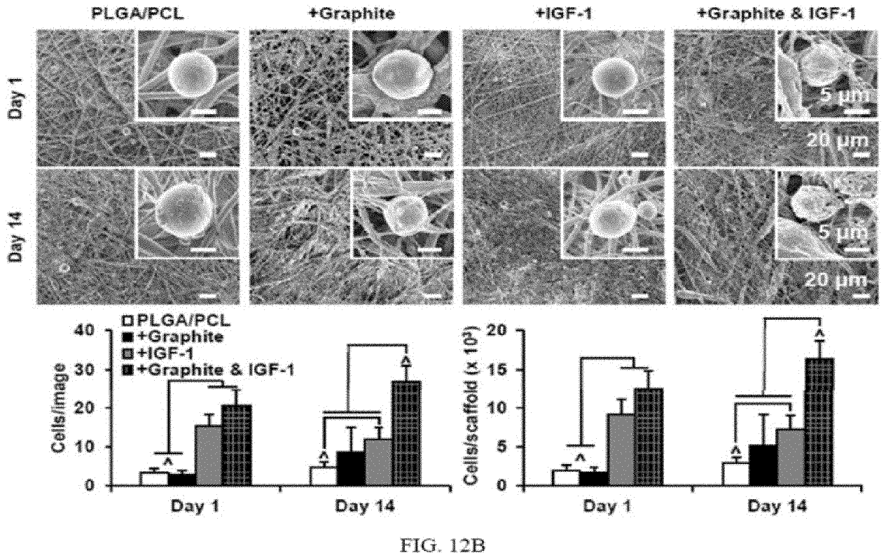

### Growth Factor Delivery and Cell Migration (FIGS. 12A and 12B).

All fibers were fabricated with no significant difference in diameter (SEM, n=50 fibers/group) (FIG. 12A). Insulin-like growth factor (IGF-1) incorporation was enhanced by the inclusion of graphite in the fibers (n=5, \hat{ }p<0.05 between groups) (FIG. 12A). While IGF-1 burst from PLGA/PCL fibers within 48 hours (n=5, *p<0.05 over time), graphite sequestered the growth factor and sustained its release over 22 days, with significantly more IGF-1 released and remaining in the graphite-containing fibers (\hat{ }p<0.05) (FIG. 12A). Combination graphite and IGF-1 therapy resulted in the greatest cell number on the fibers by day 14 of culture (n=5, 250×, \hat{ }p<0.05) (FIG. 12B).

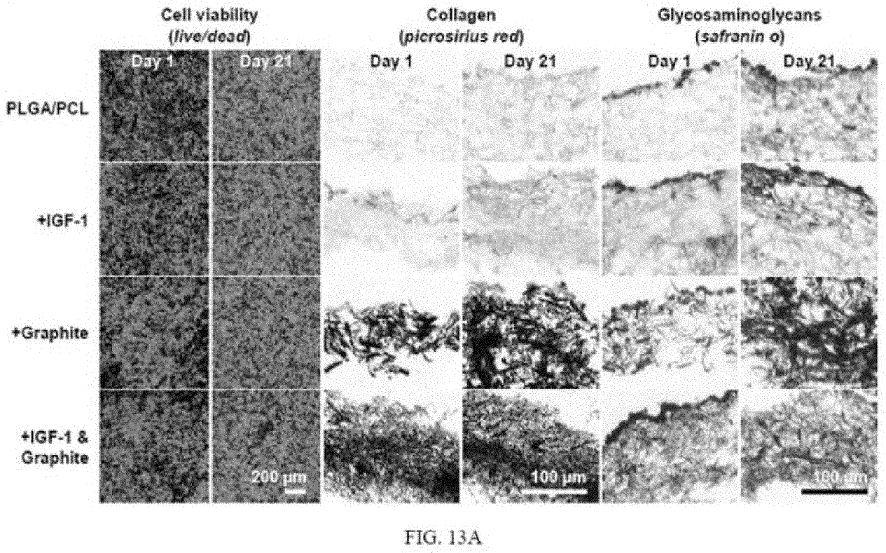

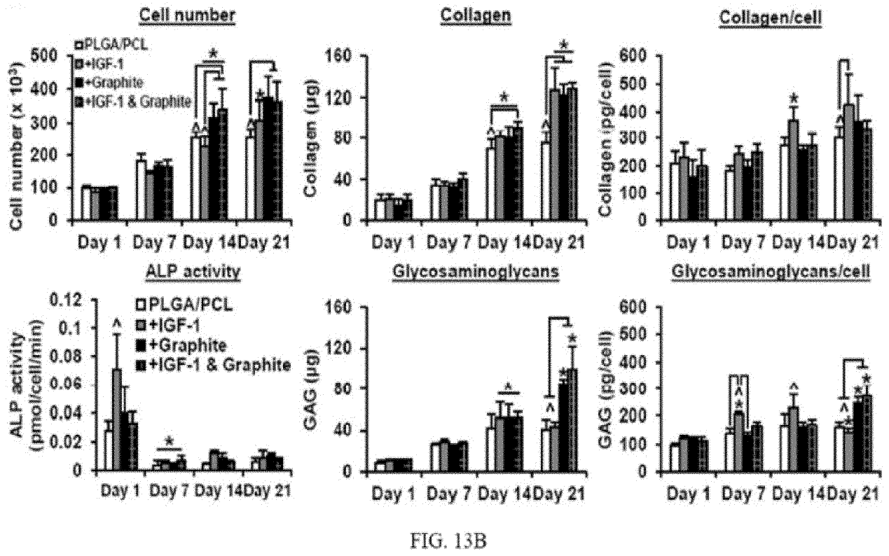

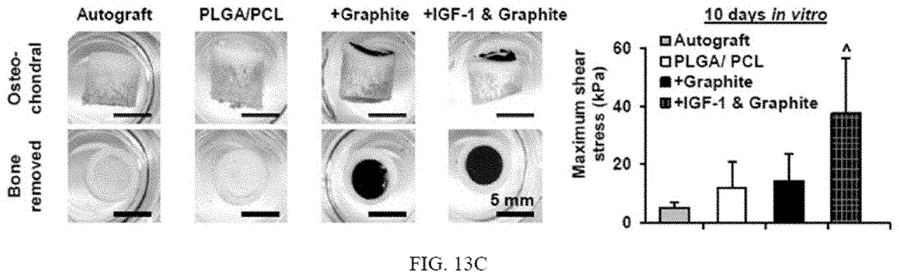

Cell Response to Combined Therapy after Direct Seeding onto Scaffolds (FIGS. 13A-13C).

Cell viability (live/dead, n=2), as well as collagen (picrosirius red, n=3) and glycosaminoglycan (GAG, safranin o, n=3) matrix deposition were visualized (FIG. 13A). Cell number increased over time (n=5, *p<0.05 over time), and graphite with or without IGF-1 enhanced proliferation by day 14 over the PLGA/PCL control (\hat{ }p<0.05 between groups) (FIG. 13B). A collagen and GAG-rich matrix was deposited in all groups, especially in the graphite-containing groups (n=5, \hat{ }p<0.05 between groups) with minimal mineralization potential measured via ALP activity (n=5) (FIG. 13B). Combining graphite with IGF-1 resulted in the greatest graft-host cartilage shear integration strength (n=6, \hat{ }p<0.05) (FIG. 13C).

### REFERENCES

Mow V C et al., Osteoarthr Cartilage, 1999
Kim Y J et al., J Biomech, 1995
Mawad D et al., Adv Func Mater, 2012
Ding X et al., Adv Healthcare Mater, 2015
Li M et al., Bioact Mater, 2018
Lamprecht C et al., ACS Appl Mater Interfaces, 2016
Reneker D H and Chun I, Nanotechnology, 1996
Kim J et al., Nat Comm, 2015
Tan A R et al., Osteoarthr Cartil, 2015
Guo B et al., Prog Polym Sci, 2013

1. A fibrous polymeric scaffold comprising electrospun fibers including a polymeric blend of poly(lactic-co-glycolic) acid (PLGA) and poly(ε-caprolactone) (PCL), in addition to graphite particles embedded in the fibers.

2. The fibrous polymeric scaffold according to claim 1, wherein the electrospun fibers of the fibrous polymeric scaffold encase a cartilage graft, to support integration of the cartilage graft with host cartilage.

3. The fibrous polymeric scaffold according to claim 2, wherein the graphite particles in the fibers impart electroactivity to the fibrous polymeric scaffold to biomimic native properties of the host cartilage operating as a mechano-transducer.

4. The fibrous polymeric scaffold according to claim 2, wherein the graphite particles embedded in the electrospun fibers of the fibrous polymeric scaffold attract cell adhesion proteins from the host cartilage.

5. The fibrous polymeric scaffold according to claim 2, wherein the graphite particles embedded in the electrospun fibers of the fibrous polymeric scaffold attract fibronectin and laminin from the host cartilage.

6. The fibrous polymeric scaffold according to claim 2, wherein the graphite particles embedded in the electrospun fibers of the fibrous polymeric scaffold enhance chondrocyte proliferation in the host cartilage.

7. The fibrous polymeric scaffold according to claim 2, wherein the host cartilage includes a zone of chondrocyte death (ZoCD), and the graphite particles embedded in the electrospun fibers of the fibrous polymeric scaffold facilitate chondrocyte repopulation in the ZoCD of the host cartilage.

8. The fibrous polymeric scaffold according to claim 2, wherein the graphite particles embedded in the electrospun fibers of the fibrous polymeric scaffold enhance glycosaminoglycan deposition in the host cartilage.

9. The fibrous polymeric scaffold according to claim 2, wherein the graphite particles embedded in the electrospun fibers of the fibrous polymeric scaffold enhance collagen production in the host cartilage.

10. The fibrous polymeric scaffold according to claim 1, wherein the graphite particles embedded in the electrospun fibers of the fibrous polymeric scaffold constitute 5% to 15% by weight in the electrospun fibers.

11. The fibrous polymeric scaffold according to claim 1, wherein the graphite particles embedded in the electrospun fibers of the fibrous polymeric scaffold enhance cell attachment to the fibrous polymeric scaffold.

12. The fibrous polymeric scaffold according to claim 1, wherein the electrospun fibers have an unaligned arrangement in the fibrous polymeric scaffold.

13. The fibrous polymeric scaffold according to claim 1, wherein in the fibrous polymeric scaffold, at least some of the electrospun fibers having the graphite particles embedded therein have a nanometer-scale diameter.

14. A method for generating a fibrous polymeric scaffold, the method comprising:
(a) dissolving poly(lactic-co-glycolic) acid (PLGA) and poly(ε-caprolactone) (PCL), individually or as a polymer blend, in a Class 3 solvent, to form a polymer solution;
(b) adding graphite particles to the polymer solution;
(c) performing electrospinning using the polymer solution in which the graphite particles have been added, to form polymeric fibers, the graphite particles constituting 5% to 15% by weight in the polymer solution employed in the electrospinning; and
(d) encasing a cartilage graft with the electrospun fibers as the fibrous polymeric scaffold, to support integration of the cartilage graft with host cartilage.

15. The method according to claim 14, wherein the graphite particles embedded in the electrospun fibers of the fibrous polymeric scaffold i) impart electroactivity to the fibrous polymeric scaffold to biomimic native properties of the host cartilage operating as a mechano-transducer, and/or ii) attract cell adhesion proteins from the host cartilage.

16. (canceled)

17. The method according to claim 14, wherein the graphite particles embedded in the electrospun fibers of the fibrous polymeric scaffold i) attract fibronectin and laminin from the host cartilage, and/or ii) enhance chondrocyte proliferation in the host cartilage.

18. (canceled)

19. The method according to claim 14, wherein the host cartilage includes a zone of chondrocyte death (ZoCD), and the graphite particles embedded in the electrospun fibers of the fibrous polymeric scaffold facilitate chondrocyte repopulation in the ZoCD of the host cartilage.

20. The method according to claim 14, wherein the graphite particles embedded in the electrospun fibers of the fibrous polymeric scaffold i) enhance glycosaminoglycan deposition in the host cartilage, ii) enhance collagen production in the host cartilage, and/or iii) enhance cell attachment to the fibrous polymeric scaffold.

21-22. (canceled)

23. The method according to claim 14, wherein the electrospun fibers encasing the cartilage graft have an unaligned arrangement.

24. The method according to claim 14, wherein at least some of the electrospun fibers encasing the cartilage graft have a nanometer-scale diameter.
